(19) United States
(12) Patent Application Publication
(10) Pub. No.: US 2011/0112445 A1
(43) Pub. Date: May 12, 2011

(54) Handpiece for Ultrasound Treatments of Human Tissue

(75) Inventor: Moreno Naldoni, Scandicci (IT)

(73) Assignee: GP Investmenti S.r.l, Montespertoli (IT)

(21) Appl. No.: 12/937,156

(22) PCT Filed: Apr. 11, 2008
(86) PCT No.: PCT/IT2008/000241

§ 371 Date: Jan. 3, 2011

(51) Int. Cl. A61N 7/00

(52) U.S. Cl. 601/2

(57) ABSTRACT

A handpiece (10) suited to carry out ultrasound treatments of human tissue to remove localised adiposity, cellulite, etc. The handpiece (10) comprises: a box-shaped body (11) presenting a basin-like concave element (13) the contour of which (CNT), in use, is rested on the skin to be treated; and two ultrasound transducers (15, 16) located on the walls of the concave element (13), the ultrasound transducers (15, 16) being inclined with respect to each other at an angle (alpha) which can be adjusted by electromechanical means. Moreover, between the two ultrasound transducers (15, 16) there is a suction mouth (14) connected to a vacuum pump. The suction mouth (14) is suited to lift a portion of skin, in such a way that two adjoining portions of skin, covered with massage oil or gel, are brought into contact with the ultrasound transducers (15, 16). Only then, the ultrasound transducers can be activated.

### TECHNICAL FIELD

The present invention concerns a handpiece, and a respective appliance provided with said handpiece, for medical and/or aesthetic treatments of adipose tissue and cellulite.

In this context the term “cellulite” is the pathology indicated as EFP (Edematous Fibrosclerotic Panniculopathy), the term used to identify the altered conditions of the subcutaneous tissues, characterised by adipose cell hypertrophy and by a certain retention of water and of fluids in general in the intercellular spaces, especially on the thighs, buttocks and abdomen.

### BACKGROUND ART

It is also known that cellulite and localised adiposities do not have a great influence on a person's physical health but, as it is an annoying imperfection and does influence the emotional state, over time many appliances and respective methods have been developed to overcome this inconvenience.

In particular, in the technique methods are known for reducing the aforesaid imperfections by means of ultrasound devices.

These devices are placed in contact with the parts of the body in which cellulite and/or adipose panniculi are present.

The ultrasounds spread through the tissues in the form of compressions and decompressions. As a result of the interaction of the ultrasounds with the biological tissues, the tissues are subjected to mechanical stress and to thermal and cavitation effects. In this context, the term “cavitation” means the phenomenon of generating “bubbles” of vapour and/or gas caused by the depression generated. These “bubbles” tend to disappear in a violent way, similar to an explosion. In this way, dispersing effects are produced within the adipose tissue.

All these effects result in a destruction or reduction of the adipose tissue and of the cellulite.

However, the ultrasound appliances currently available on the market are unable to obtain appreciable results as regards a rapid reduction of adiposities or of cellulite, or they present risk factors connected to the focalisation of the ultrasound beam or the generation of ultrasound frequencies considered unsafe for use on the human body.

The handpiece, and the respective appliance using the handpiece, to which the present invention refers, are ultrasound instruments able to exert an efficacious action on the tissues to be treated.

### DISCLOSURE OF INVENTION

Therefore, according to the present invention an ultrasound handpiece and a respective appliance that uses said handpiece are made in accordance with the enclosed claims.

### BRIEF DESCRIPTION OF THE DRAWINGS

The present invention will now be described with reference to the enclosed drawings, which illustrate some examples of embodiments without limitation, wherein:

FIG. 1 illustrates a three-dimensional front view of an ultrasound handpiece according to the invention;

FIG. 2 shows a three-dimensional rear view of the ultrasound handpiece in FIG. 1;

FIG. 3 illustrates an appliance for the complete treatment of cellulite; said appliance contemplates the use of a handpiece for ultrasound treatments and of a handpiece for cutaneous massage.

### BEST MODE FOR CARRYING OUT THE INVENTION

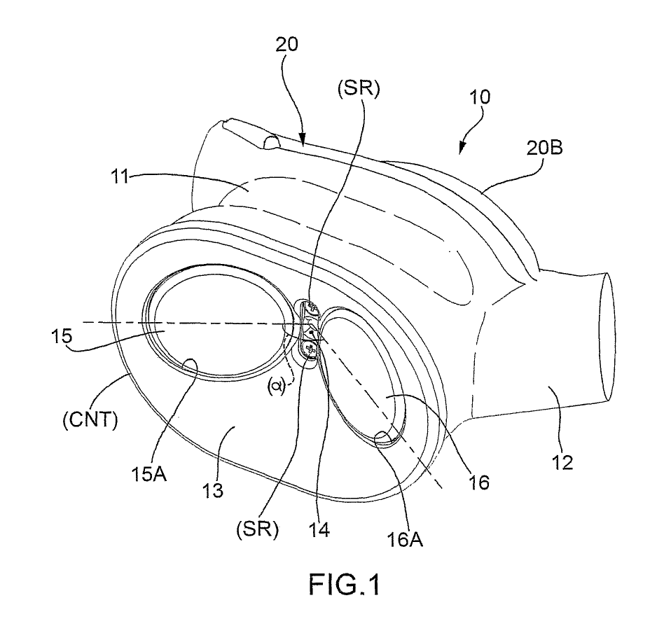

In FIG. 1, the reference 10 indicates, on the whole, an ultrasound handpiece according to the present invention.

As shown in the enclosed figures, the ultrasound handpiece 10 comprises a box-shaped body 11, hollow on the inside, suited to house the electronic devices (not shown) necessary for the operation of the handpiece itself.

The box-shaped body 11 extends laterally with a sleeve 12 obtained enbloc with the box-shaped body 11 itself. Preferably, but not necessarily, the box-shaped body 11 and the sleeve 12 are made in a single moulding operation of plastic material.

As shown in FIG. 1, fixed to the box-shaped body 11 by means of two screws (SR) is a basin-like concave element 13 the contour of which (CNT), in this particular embodiment, is substantially elliptical in shape.

Moreover, in the embodiment in FIG. 1, between the two aligned retaining screws (SR) there is a suction mouth 14 positioned in the centre of the substantially elliptical contour (CNT). The suction mouth 14 is connected to a vacuum pump (electro-pneumatic means) (not illustrated).

On opposite sides with respect to the suction mouth 14 and to the two screws (SR) are placed two ultrasound transducers 15, 16 in the shape of flat circular pellets.

In the embodiment shown in FIG. 1, each ultrasound transducer 15, 16 presents a peripheral ring-shaped collar (not shown) and is housed in a respective circular seat 15A, 16A.

Moreover, each ultrasound transducer 15, 16 is held fixed with respect to the box-shaped body 11 by the concave element 13 fitted on the box-shaped body 11 which presses the respective peripheral ring-shaped collar against the box-shaped body 11 itself.

Since the arrangement of the ultrasound transducers 15, 16 follows the shape of the walls of the concave element 13, these ultrasound transducers 15, 16 are inclined with respect to each other at an angle (α) between 90° and 170°, preferably 130°.

In a further embodiment not illustrated, the possibility is considered of varying, by means of suitable electromechanical systems, the relative angle between the two ultrasound transducers 15, 16 (from 0° to 180°), so as to adapt the geometry of the active part of the handpiece to the thickness of the adipose layer and/or of the part of the human body that is to be treated.

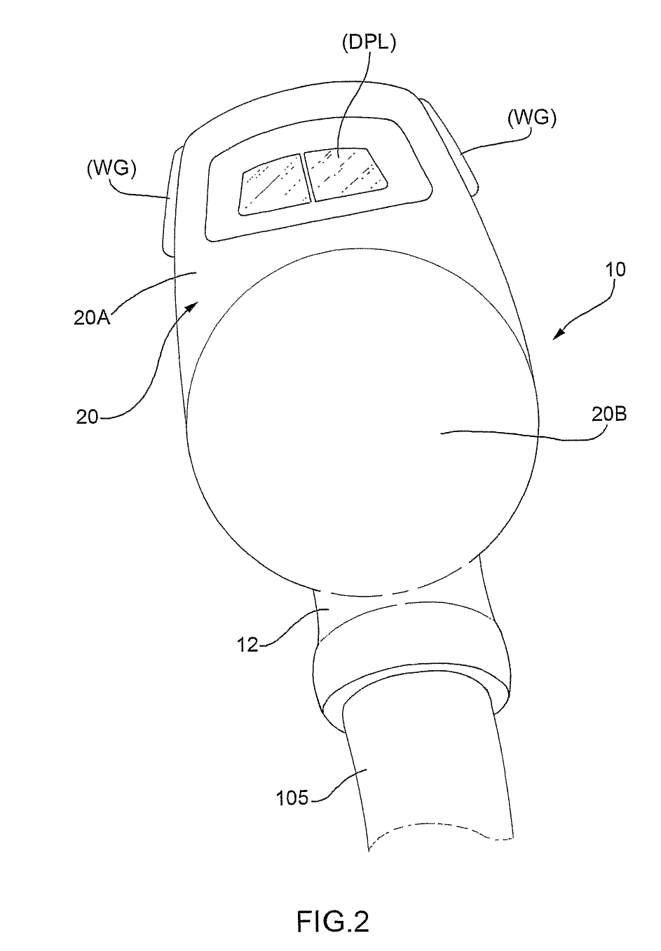

Instead, FIG. 2 shows a three-dimensional rear view of the ultrasound handpiece in FIG. 1.

On the part opposite the concave element 13, the box-shaped body 11 is closed by a cover 20 comprising a first portion 20A, which is substantially flat, and a hemispherical second portion 20B.

As also shown in FIG. 2, on the first portion 20A is a display (DPL) on which appear the data concerning the treatment that is being carried out (see below).

On the sides of the first portion 20A are two buttons (WG) which, if pressed by the operator's fingers, allow him to control the treatment cycle.

In particular, one button (WG) enables him to start and stop the treatment, while the second button (WG) enables him to suspend the pump suction temporarily so as to release the tissue, allowing easy movement of the handpiece.

These two buttons (WG) allow the operator to control a treatment appliance (see below), during treatment, without having to reach the main control panel of the appliance (see below).

This characteristic has two important positive consequences:
(1) the operator can carry out the treatment and concentrate on the therapy without having to worry about the machine position or the eventual inability to reach the main control panel; and
(2) it guarantees a long life of the main control panel since it reduces the possibility of its coming in contact with substances that could degrade its operation.

The second portion 20B is preferably made enbloc with the first portion 20A. The construction of the cover 20 is achieved advantageously, but not necessarily, by means of a moulding procedure of a plastic material, similar to or the same as the one with which the box-shaped body 11 is made.

In addition, the second portion 20B advantageously presents a preferably hemispherical conformation, or more generally convex, to allow the operator to hold the handpiece 10 with ease in his hand.

In use, after having spread oil or gel (substances which allow the correct propagation of the ultrasound and which may contain active principles that assist the treatment), preferably oil, on the skin of the person who is about to be subjected to the treatment, the operator rests the contour (CNT) of the concave element 13 on the skin, starting operation of the handpiece 10.

In the area corresponding to the suction mouth 14 a vacuum is created (thanks to the aforementioned connection of the suction mouth 14 to a vacuum pump) which lifts a piece of tissue, this tissue rests against the ultrasound transducers 15, 16 which begin to perform the treatment on the raised skin.

Obviously, as the handpiece 10 is manually moved by the operator, always different parts of human tissue are treated in sequence until the whole area affected by the adipose phenomenon or by EFP has been treated.

Moreover, it has been found in experiment that, by treating the raised tissue simultaneously with two ultrasound beams from the transducers 15, 16, notable advantages may be obtained as regards the efficacy and safety of the treatment. In this way, in fact, the crossed action of the ultrasound beams is concentrated on the affected portion of tissue and, therefore, the energy is concentrated where it is useful without needlessly irradiating the organs below. The treatment is therefore safer and more efficacious.

The electronic devices (not shown) present in the handpiece 10 and/or in the machine body (see below) are suited to implement, in the ultrasound transducers 15, 16, cyclic treatments in which the two devices are led to work together or alternately, at a variable frequency between 4 kHz and 100 kHz, modulating a carrier of 1 MHz.

The two ultrasound transducers 15, 16 can also work in swept mode, that is with a frequency that increases over time between the two above-mentioned limits.

Moreover, in an embodiment not illustrated, the angle (α) between the two ultrasound transducers 15, 16 is varied between 0° and 180° by means of an electromechanical device (not illustrated) present in the box-shaped body 11, according to the thickness of the adipose layer and/or to the part of the human body that is to be treated.

In addition, in the handpiece 10 the emission of ultrasound through the two ultrasound transducers 15, 16 is activated only when the fold of skin has been lifted with the vacuum pump corresponding to the suction mouth 14.

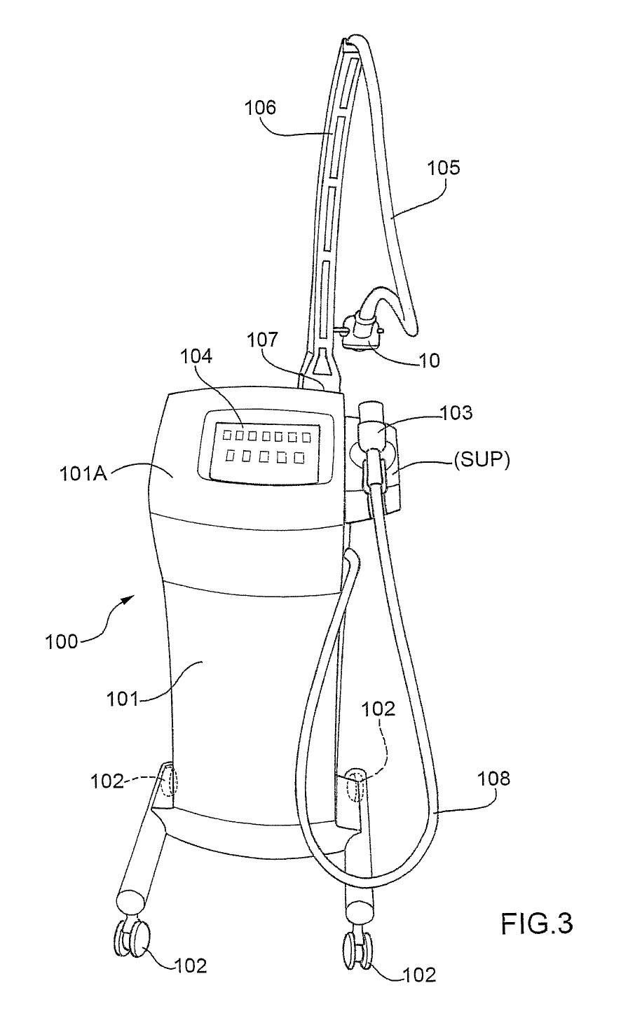

FIG. 3 shows an appliance 100 for the complete treatment of cellulite.

The appliance 100 comprises a removable machine-body 101, mounted on wheels 102.

Inside the machine-body 101 are most of the electronic devices (not shown) suited to activate a handpiece 10 for ultrasound treatments (of the type already described with reference to FIGS. 1, 2), and a handpiece 103 for cutaneous massage, for example like the one illustrated and claimed in the application for a European patent EP-1 610 748 (in the name of the same Applicant), the content of which must be considered an integral part of this description. In particular the handpiece 103 uses an elastomer membrane, to which is given a pulsating movement, to perform cutaneous massage.

Moreover, housed in the machine-body 101 is the vacuum pump (not shown) for sucking up the portions of tissue by means of the suction mouth 14.

On the top surface 101A of the machine-body 103 is located a display 104 of the traditional type, or of the “touch screen” type, with which the operator gives the necessary commands to the handpieces 10, 103 to perform the desired treatments on the parts of the body affected by the presence of cellulite and/or of adipose tissue.

Massaging with the handpiece 103 on the area treated previously with ultrasound using the handpiece 10 brings the fat particles broken up by the ultrasound towards the lymphatic stations pertaining to the treated area. So a real lymphatic drainage is performed. The dispersed fat can then be disposed of through the normal metabolic processes. In act the fat particles must not continue to remain in the area that they occupied previously, but they must be sent into lymphatic circulation so as to be eliminated from the organism.

As also shown in FIG. 3, a first cable tube 105 connects the machine-body 101 to the ultrasound handpiece 10. Inside the first cable tube 105 are the cables for the electricity supply to the handpiece, and for exchanging information between the boards present on the handpiece 10 itself and an electronic control unit (not shown) housed in the machine-body 101. The cable tube 105 is connected to the handpiece by means of the sleeve 12 (FIG. 2).

A portion of the cable tube 105 is held up by a trellis support 106 presenting an articulation 107 provided with elastic means (not shown) that tend to bring the support 106 back into erect position if stimulated during the unwinding of the cable tube 105. The presence of the support 106 is necessary to prevent the first cable tube 105, during use, from becoming snarled, or even tangled with a second cable tube 108 which contains the cables (not shown) for the electricity supply to the handpiece 103, and for exchanging information between the boards present on the handpiece 103 itself and the electronic control unit housed in the machine-body 101. Moreover, when in rest position, the handpiece 103 rests on a support (SUP) projecting from the side of the machine-body 101.

The main advantages of the handpiece described above are the following:
“optimum” positioning of the tissues so that they can receive ultrasound radiation when they are raised to form a “fold”; in this case the radiation is safer and more efficacious, as most of the ultrasound energy is in a direction perpendicular to the adipose parts, but not to the internal organs;
crossing of two ultrasound beams through a synergic and doubly effective action;
modulation of a carrier with a frequency of 1 MHz, allowed by the regulations in force and therefore considered safe for applications on the human body with low frequency pulse trains (10 to 100 kHz) suited to break up the adipose cells, in this way combining the disruptive action of the low frequency pulse trains (10 to 100 kHz) on the superficial tissue layers with the safety guaranteed by the 1 MHz carrier which does not allow damage to the deep-seated organs.

Further advantages of the appliance comprising an ultrasound handpiece and a handpiece for cutaneous massage are the following:
the action of the low-frequency ultrasounds is followed by a lymphatic drainage massage which assists the elimination of the fat particles broken up by the cavitation effect and of any excess fluids;
the handpiece for cutaneous massage allows lymphatic drainage massage integrated in the machine, without having to resort to manual massage and/or to the use of another mechanical massage device;
lymphatic drainage is accomplished by means of the special vibrating elastomer membrane (as in the application for a patent EP-1 610 748), which performs a complete, efficacious and painless movement on the tissue;
by using the handpiece with an elastomer membrane it is also possible to open the lymphatic stations before lymphatic drainage, thus facilitating the flow, through the lymphatic system, of the adipose cells destroyed by the ultrasound action.

1. Handpiece (10) suited to carry out ultrasound treatments of human tissue to remove localised adiposity, cellulite, etc.
handpiece (10) comprising at least one ultrasound transducer (15, 16) and an element (20) for holding the handpiece (10) itself;
handpiece (10) characterised in that it comprises:
box-shaped body (11) presenting a basin-like concave element (13) the contour of which (CNT), in use, rests on the skin to be treated;
two ultrasound transducers (15, 16) placed on the walls of said concave element (13), said ultrasound transducers (15, 16) being inclined with respect to each other at an angle (α); and in that between said two ultrasound transducers (15, 16) there is a suction mouth (14) connected to electro-pneumatic means, said suction mouth (14) being suited to lift a fold of tissue, in such a way that two adjoining portions of tissue, spread preferably with massage oil or gel, are brought into contact with the ultrasound transducers (15, 16).

2. Handpiece (10) as in claim 1, characterised in that the angle (α) is between 90° and 170°, preferably 130°.

3. Handpiece (10) as in claim 1, characterised in that the contour (CNT) of the basin-like concave element (13) is substantially elliptical in shape.

4. Handpiece (10) as in claim 1, characterised in that each of the two ultrasound transducers (15, 16) is in the shape of a flat circular pellet.

5. Handpiece (10) as in claim characterised in that the two emitted ultrasound beams cross over each other.

6. Handpiece (10) as in claim 1, characterised in that the relative angle of the two ultrasound transducers (15, 16) is varied between 0° and 180°, using electromechanical means, according to thickness of the adipose layer and/or of the part of the human body that is to be treated.

7. Handpiece (10) as in claim 1, characterised in that the ultrasound emission through the two ultrasound transducers (15, 16) is activated only when the fold of skin has been raised with electro-pneumatic means.

8. Appliance (100) characterised in that it comprises at least one ultrasound handpiece (10) according to claim 1.

9. Appliance (100) according to claim 8, characterised in that it comprises an ultrasound handpiece (10) and a handpiece (103) for carrying out cutaneous massage, suited to drain the fat particles previously broken up by the ultrasound towards the lymphatic stations.

10. Appliance (100) according to claim 9, characterised in that said handpiece (103) comprises an elastomer membrane, to which is given a pulsating movement, to perform cutaneous massage.
